(19) United States
(12) Patent Application Publication
(10) Pub. No.: US 2022/0132882 A1
(43) Pub. Date: May 5, 2022

(54) Herbal Tea Products and Methods

(71) Applicant: Rene O. Guzman, Houston, TX (US)

(72) Inventor: Rene O. Guzman, Houston, TX (US)

(21) Appl. No.: 17/461,942

(22) Filed: Aug. 30, 2021

Related U.S. Application Data
(60) Provisional application No. 63/204,875, filed on Nov. 2, 2020.

(51) Int. Cl. A23F 3/34; A61K 36/53; A61K 36/534; A61K 36/537; A61K 36/484; A61K 36/30; A61K 36/236; A61K 36/61; A61K 36/68; A61K 36/185; A61K 36/34; A61K 36/28; A61K 9/00

(52) U.S. Cl. CPC A23F 3/34; A61K 2236/15; A61K 36/534; A61K 36/537; A61K 36/484; A61K 36/30; A61K 36/236; A61K 36/61; A61K 36/68; A61K 36/185; A61K 36/34; A61K 36/28; A61K 9/0053; A61K 2236/37; A61K 2236/331; A61K 36/53

(57) ABSTRACT

An herbal tea product is provided to provide relief from lung and breathing conditions such as asthma. The herbal tea product may include oregano, peppermint, OSHA root, lungwort, eucalyptus, plantain leaf, chaparral, elecampane, lobelia, sage, licorice root, and thyme. The herbal ingredients may be included in equal amounts, such as 5 ounces. The OSHA root, elecampane, and/or licorice root may be crushed. Methods of making and using the herbal tea product are also disclosed. The herbal tea product may be placed into tea bags, and prepared for human consumption by placing the tea bag with herbal tea product in hot water that has been brought to a boil. The herbal tea product may be consumed in appropriate amounts, such as 16 ounces, twice a day for two weeks, and then once a day thereafter.

### RELATED APPLICATIONS

This application claims the benefit of U.S. Provisional Application No. 63/204,875, filed Nov. 2, 2020, the contents of which are fully incorporated herein by reference.

### BACKGROUND OF THE INVENTIONS

### 1. Field of the Inventions

The present inventions generally pertain to tea, and more particularly to unique blends of tea and related methods that are useful to provide relief from lung or breathing conditions such as asthma.

### 2. Description Of The Related Art

There are many known compositions and treatments for those who suffer from lung or breathing conditions such as asthma. Examples of such known compositions and treatments include long-term control medications, such as inhaled corticosteroids medications, and quick-relief inhalers, which contain a fast-acting medication albuterol and are sometimes called rescue inhalers. However, these known compositions and treatments have various shortcomings. As will become apparent from the description and explanation set forth below, the present inventions overcome deficiencies with existing breathing solutions for lung and breathing conditions such as asthma.

### SUMMARY OF THE INVENTIONS

In one aspect, the present inventions may include an herbal tea product that may comprise oregano, peppermint, lungwort, sage and licorice root. Another feature of this aspect of the present inventions may be that the herbal tea product may further include at least one of OSHA root, eucalyptus, plantain leaf, chaparral, elecampane, lobelia, and thyme. Another feature of this aspect of the present inventions may be that the herbal tea product may include equal amounts of at least two of oregano, peppermint, OSHA root, lungwort, eucalyptus, plantain leaf, chaparral, elecampane, lobelia, sage, licorice root, and thyme. Another feature of this aspect of the present inventions may be that the equal amounts of at least two of oregano, peppermint, OSHA root, lungwort, eucalyptus, plantain leaf, chaparral, elecampane, lobelia, sage, licorice root, and thyme equal approximately five ounces. Another feature of this aspect of the present inventions may be that the licorice root may comprise crushed licorice root. Another feature of this aspect of the present inventions may be that the OSHA root comprises crushed OSHA root, and the elecampane may comprise crushed elecampane.

In another aspect, the present inventions may include an herbal tea product comprising: at least one of oregano, peppermint, and OSHA root; at least one of lungwort, eucalyptus, and plantain leaf; at least one of chaparral, elecampane, and lobelia; and at least one of sage, licorice root, and thyme.

In another aspect, the present inventions may include a method of making an herbal tea product comprising: measuring a portion of oregano; measuring a portion of peppermint, measuring a portion of lungwort; measuring a portion of sage; measuring a portion of licorice root; and mixing the measured portions of oregano, peppermint, lungwort, sage, and licorice root. Another feature of this aspect of the present inventions may be that method may further include: measuring a portion of at least one of OSHA root, eucalyptus, plantain leaf, chaparral, elecampane, lobelia, and thyme; and mixing the measuring portions of at least one of OSHA root, eucalyptus, plantain leaf, chaparral, elecampane, lobelia, and thyme with the mixture of oregano, peppermint, lungwort, sage, and licorice root. Another feature of this aspect of the present inventions may be that the measured portions of at least two of oregano, peppermint, OSHA root, lungwort, eucalyptus, plantain leaf, chaparral, elecampane, lobelia, sage, licorice root, and thyme may be in equal amounts. Another feature of this aspect of the present inventions may be that the measured amounts of at least two of oregano, peppermint, OSHA root, lungwort, eucalyptus, plantain leaf, chaparral, elecampane, lobelia, sage, licorice root, and thyme may be five ounces. Another feature of this aspect of the present inventions may be that the method may further include crushing the licorice root. Another feature of this aspect of the present inventions may be that the method may further include crushing at least one of the OSHA root and the elecampane. Another feature of this aspect of the present inventions may be that the method may further include placing a portion of the mixture in a tea bag.

In another aspect, the present inventions may include a method of using an herbal tea product comprising oregano, peppermint, lungwort, sage, and licorice root, comprising: bringing a volume of water to a gentle boil; placing a tea bag containing the herbal tea product in the water; setting the water and tea bag aside until the color of the tea water has a brown color; pouring a portion of the colored tea water into a container; and drinking the tea water from the container. Another feature of this aspect of the present inventions may be that the portion of colored tea water that is poured into the container may amount to approximately 16 ounces. Another feature of this aspect of the present inventions may be that the above steps may be repeated twice each day for two weeks and two containers of colored tea water may be consumed each day for two weeks. Another feature of this aspect of the present inventions may be that the above steps may be repeated once each day thereafter and one container of colored tea water may be consumed on each such day. Another feature of this aspect of the present inventions may be that the licorice root in the herbal tea product may be crushed. Another feature of this aspect of the present inventions may be that the herbal tea product may further include at least one of OSHA root, eucalyptus, plantain leaf, chaparral, elecampane, lobelia, and thyme. Another feature of this aspect of the present inventions may be that any OSHA root or elecampane in the herbal tea product may be crushed.

Other features, aspects and advantages of the present inventions will become apparent from the following discussion and detailed description.

### BRIEF DESCRIPTION OF THE DRAWINGS

FIG. 1 is a flow chart illustrating a specific embodiment of a method of making an herbal tea product.

FIG. 2 is schematic illustration showing containers of various herbs that may be used in combination to form one or more specific embodiments of herbal tea products in accordance with the present inventions.

FIG. 3 illustrates measured amounts of herb ingredients being placed into mixing bowl.

FIG. 4 illustrates the mixing bowl with a mixed herbal tea product contained therein.

FIG. 5 illustrates a measured portion of the herbal tea product being moved from the mixing bowl into an empty tea bag.

While the inventions will be described in connection with the preferred embodiments, it will be understood that the scope of protection is not intended to limit the inventions to those embodiments. On the contrary, the scope of protection is intended to cover all alternatives, modifications, and equivalents as may be included within the spirit and scope of the inventions as defined by the appended claims.

### DETAILED DESCRIPTION OF THE INVENTION

The present inventions encompass various compositions of unique tea mixtures and methods of making and using same that are useful to provide relief from lung or breathing conditions such as asthma. In a specific embodiment, the present inventions may comprise an herbal tea product that may comprise the following ingredients: oregano, peppermint, lungwort, sage, and licorice root. In another specific embodiment, the present inventions may comprise an herbal tea product that may include: at least one of oregano, peppermint, and OSHA root; at least one of lungwort, eucalyptus, and plantain leaf; at least one of chaparral, elecampane, and lobelia; and at least one of sage, licorice root, and thyme.

In another specific embodiment, the present inventions may comprise an herbal tea product that may comprise the following ingredients: oregano, peppermint, OSHA root, lungwort, eucalyptus, plantain leaf, chaparral, elecampane, lobelia, sage, licorice root, and thyme. It has been discovered that this unique blend of herbs has a synergistic impact on the ability to provide breathing relief to those suffering from lung and breathing conditions, such as asthma. The various ingredients and their characteristics are set forth below. While these herbs and their characteristics are known, the unique herbal product blends and methods in accordance with the present inventions, and their efficacy in the treatment of lung and breathing conditions, such as asthma, have not been heretofore known.

Oregano is part of the mint family (also known as the Lamiaceae family). Oregano has been used for its flavor and medicinal qualities. In the kitchen, oregano is primarily used to flavor sauces, flavor dinner rolls, and marinade meat. Oregano is loaded with calcium and Vitamin K, and can help with the growth of bones and teeth. Additionally, oregano is considered a rich source of antioxidants that can help naturally fight infection and boost the immune system.

Peppermint has been used for centuries as a medicinal herb. Common applications for peppermint include relief from indigestion and motion sickness. Peppermint is also commonly used in mouth products (like toothpaste and mouthwash) because it can reduce the growth of bacteria. In the case of providing natural lung support, peppermint can both sooth and relax the body. Allergens and foreign bodies (like those inhaled when a person is around smoke) can cause a person's nasal tract (including throat, nose, bronchi, and lungs) to swell and become congested. Peppermint acts as a natural antihistamine. Additionally, peppermint has high levels of menthol offer a cooling relief.

Osha root is native to the Rocky Mountains. Osha root grows in the upland meadows and ravines. Osha root, which is also known as bear root, is marked by a perennial white flower that grows in tightly clustered balls. Osha root is the root of the osha plant, which offers medicinal benefits. For thousands of years, Native Americans in the area have considered it sacred and used it to treat a wide range of respiratory issues, including colds and coughs. Not only can Osha root help relieve congestion, but it can also increase circulation in the lung. This can make it easier to take deep breaths.

Lungwort is known to help the respiratory system. Lungwort is a flowering plant that resembles infected lung tissue. Lungwort grows in damp, natural environments. Lungwort is extremely sensitive to toxins, so if it is growing in a given area then it is an indicator of a healthy ecosystem. Lungwort is high in antioxidants. The primary use of lungwort is for respiratory relief. The leaves of the lungwort plant are known to offer medicinal benefits. Once the leaves are harvested and dried, they can then be used in medicinal supplements or steeped in hot water to make a tea.

Eucalyptus is known for its ability to soothe sunburns and feed Koala bears. But this Australian plant can also do wonders for the inside of the human body. Eucalyptus has a refreshing aroma that has been found to promote good respiratory health. Additionally, eucalyptus can soothe the throat when raw and sore, help fight congestion, and reduce the need to cough. Eucalyptus is often found in cough drops. Diluted eucalyptus oil can also be added to teas and applied topically to the skin.

Plantain leaf has been used to soothe coughs and improve respiratory function. Despite the similarity in name, plantain leaves are not the leaves found growing near the banana-like fruit. Rather, plantain leaf is a green, weed-like plant that grows close to the ground. Plantain leaf is typically found in balms that are rubbed onto the chest.

Chaparral is a bright yellow flowering plant. It grows in the deserts of southwest North America. Chaparral is high in nordihydroguaiaretic acid (also known as NGDA). Chaparral is a powerful antioxidant. Chaparral can help detoxify the respiratory system, giving lungs the ability to heal naturally. Additionally, chaparral can reduce congestion, coughing, and swelling, which can help the throat and nasal passage heal.

Elecampane has been cited for its use in treating respiratory issues by early Greek, Roman, and Chinese doctors. Generally, the root of the elecampane plant is utilized. Elecampane can be consumed in capsules, tinctures, and teas. Elecampane has two active compounds that can offer natural lung support. The first is inulin, a compound that soothes your bronchial passage. The second is alantolactone, which has anti-inflammatory action and also reduces mucous secretions.

Lobelia can offer a wide range of health benefits including detoxification, controlling hunger cravings, and relieving respiratory distress. This is partially due to alkaloid, a compound which can thin mucous and break up congestion.

Sage functions as a natural diuretic, stimulating the kidneys and liver, which means that more toxins are flushed through the body by way of urination. In the same way, sage also helps promote kidney and liver health. Since sage tea triggers sweating, the body is further detoxified through the skin, which stimulates the kidneys and liver.

Licorice root has a wide range of health benefits such as detoxifying the body, soothing spasms, easing menstrual cramps, raising blood pressure, eliminating respiratory infections, reducing inflammation, treating stomach disorders, protecting the skin and strengthening the hair.

Thyme is indigenous to the Mediterranean region. Wild thyme grows in the Levant where it might have been st cultivated. Interesting health benefits of thyme include its ability to relieve stress, reduce respiratory issues, improve heart health, boost the strength of the immune system, protect against chronic diseases, stimulate blood flow, and prevent fungal infections.

It has been discovered that mixing these herbs together in a tea and consuming same over time will result in improved breathing without an inhaler over time for those suffering from breathing conditions, such as asthma.

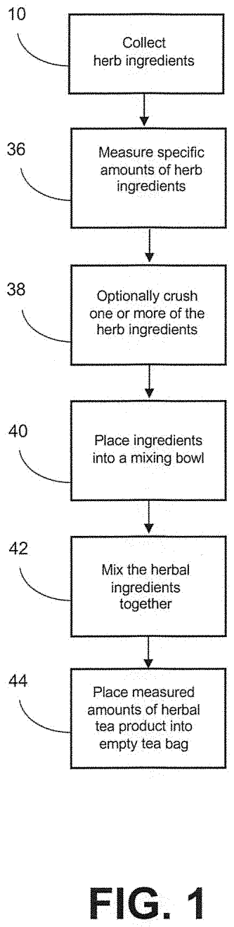

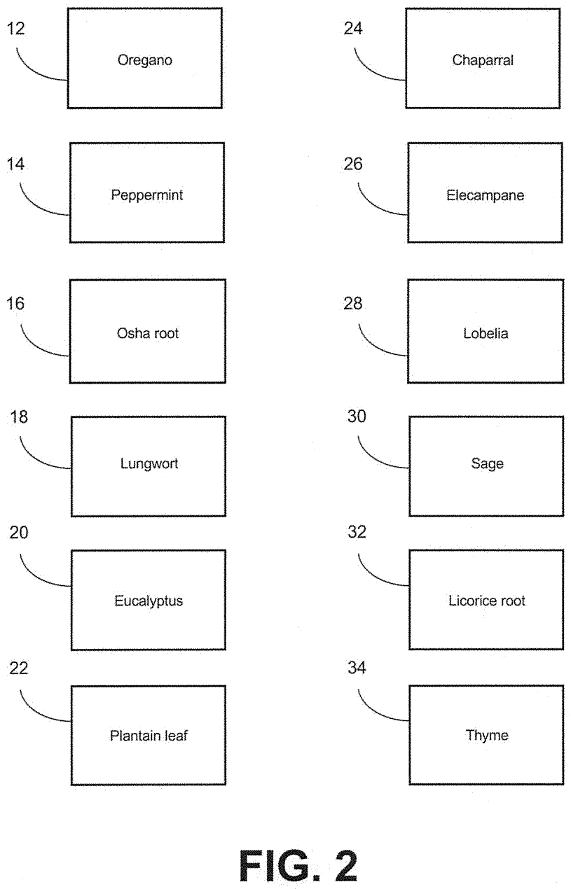

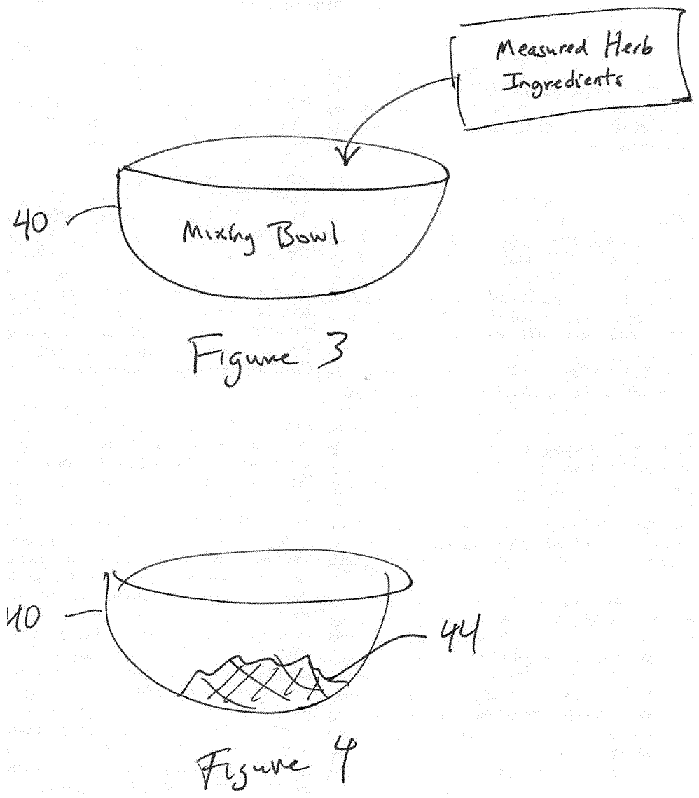

A method of manufacturing an herbal tea product in accordance with the present inventions will now be described in connection with FIGS. 1-4. As shown in FIG. 1, which is a flow chart setting forth a specific embodiment of example steps that can be taken to make an herbal tea product in accordance with the present inventions, at step 10, some or all of the above-discussed herbs are collected. As shown in FIG. 2, in a specific embodiment, the herbs to be collected may include containers of oregano 12, peppermint 14, OSHA root 16, lungwort 18, eucalyptus 20, plantain leaf 22, chaparral 24, elecampane 26, lobelia 28, sage 30, licorice root 32, and thyme 34. Referring again to FIG. 1, next, at step 36, the various herbal ingredients to be used are measured, and at step 40 placed into a mixing bowl 40, a shown in FIG. 3. For example, in a specific embodiment, equal amounts of each herb to be used in the herbal tea product may be measured. In another specific embodiment, five ounces of each herb may be measured.

Referring again to FIG. 1, in a specific embodiment, at step 38, one or more of the measured amounts of herbs may be crushed. This crushing may be done before or after placing the herb into the mixing bowl 40. In a specific embodiment, at least one of the following herbs, preferably the roots, will be crushed into small pieces: OSHA root, elecampane, and licorice root.

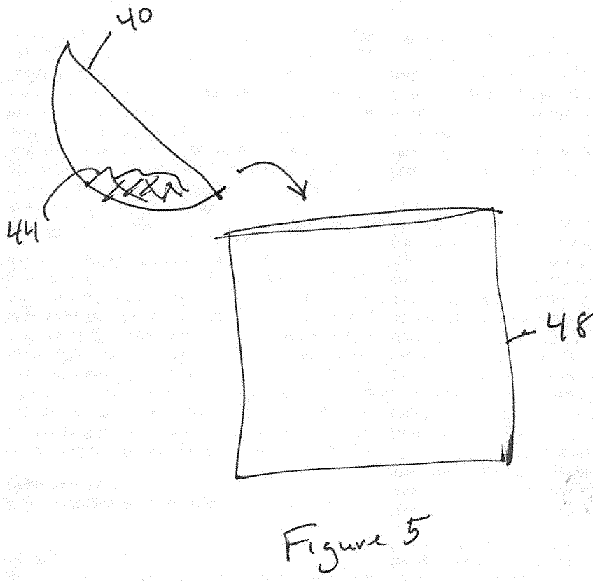

Next, at step 42, all of the herbs that have been placed into the mixing bowl 42, some of which may have been crushed, are thoroughly mixed together to form an herbal tea product 44, as shown in FIG. 4. Next, at step 46, in a specific embodiment, a predetermined amount of the herbal tea product 44 (e.g., one teaspoon) may be placed into an empty tea bag 48, as shown in FIG. 5. Next, bring some water (e.g., 8 ounces) to a gentle boil, add the tea bag 48 to the hot water, and set aside until the color of the tea water has a brown color. Next, the herbal tea may be served in a cup (e.g., an 8 ounce cup) and a person may drink the herbal tea. In a specific embodiment, the tea is preferably served hot, not cold, for the best results.

In a specific embodiment, a method of using an of the herbal tea products of the present inventions may be to drink two glasses (e.g., 16 ounces per glass) a day for two weeks, and then each day thereafter take 1 glass (e.g., 16 ounces) per day. Potential side effects may include constipation and headaches. Drinking the herbal tea products of the present inventions are beneficial to persons with asthma or other lung problems, as they will help to naturally clear the person's lungs, improve lung health, and help the person to breathe easier and more comfortably.

It is to be understood that the inventions disclosed herein are not limited to the exact details of construction, operation, exact materials or embodiments shown and described. Although specific embodiments of the inventions have been described, various modifications, alterations, alternative constructions, and equivalents are also encompassed within the scope of the inventions. Although the present inventions may have been described using a particular series of steps, it should be apparent to those skilled in the art that the scope of the present inventions is not limited to the described series of steps. The specification and drawings are, accordingly, to be regarded in an illustrative rather than a restrictive sense. It will be evident that additions, subtractions, deletions, and other modifications and changes may be made thereunto without departing from the broader spirit and scope of the inventions as set forth in the claims set forth below. Accordingly, the inventions are therefore to be limited only by the scope of the appended claims. None of the claim language should be interpreted pursuant to 35 U.S.C. 112(f) unless the word “means” is recited in any of the claim language, and then only with respect to any recited “means” limitation.

1. An herbal tea product comprising:
oregano;
peppermint;
lungwort;
sage; and
licorice root.

2. The herbal tea product of claim 1, wherein the herbal tea product further includes at least one of OSHA root, eucalyptus, plantain leaf, chaparral, elecampane, lobelia, and thyme.

3. The herbal tea product of claim 2, wherein the herbal tea product includes equal amounts of at least two of oregano, peppermint, OSHA root, lungwort, eucalyptus, plantain leaf, chaparral, elecampane, lobelia, sage, licorice root, and thyme.

4. The herbal tea product of claim 3, wherein the equal amounts of at least two of oregano, peppermint, OSHA root, lungwort, eucalyptus, plantain leaf, chaparral, elecampane, lobelia, sage, licorice root, and thyme equal approximately five ounces.

5. The herbal tea product of claim 1, wherein the licorice root comprises crushed licorice root.

6. The herbal tea product of claim 2, wherein the OSHA root comprises crushed OSHA root, and the elecampane comprises crushed elecampane.

7. An herbal tea product comprising:
at least one of oregano, peppermint, and OSHA root;
at least one of lungwort, eucalyptus, and plantain leaf;
at least one of chaparral, elecampane, and lobelia; and
at least one of sage, licorice root, and thyme.

8. A method of making an herbal tea product comprising:
measuring a portion of oregano;
measuring a portion of peppermint, measuring a portion of lungwort;
measuring a portion of sage;
measuring a portion of licorice root; and
mixing the measured portions of oregano, peppermint, lungwort, sage, and licorice root.

9. The method of claim 8, further including:
measuring a portion of at least one of OSHA root, eucalyptus, plantain leaf, chaparral, elecampane, lobelia, and thyme; and mixing the measuring portions of at least one of OSHA root, eucalyptus, plantain leaf, chaparral, elecampane, lobelia, and thyme with the mixture of oregano, peppermint, lungwort, sage, and licorice root.

10. The method of claim 9, wherein the measured portions of at least two of oregano, peppermint, OSHA root, lungwort, eucalyptus, plantain leaf, chaparral, elecampane, lobelia, sage, licorice root, and thyme are in equal amounts.

11. The method of claim 10, wherein the measured amounts of at least two of oregano, peppermint, OSHA root, lungwort, eucalyptus, plantain leaf, chaparral, elecampane, lobelia, sage, licorice root, and thyme is five ounces.

12. The method of claim 8, further including crushing the licorice root.

13. The method of claim 9, further including crushing at least one of the OSHA root and the elecampane.

14. The method of claim 9, further including placing a portion of the mixture in a tea bag.

15. A method of using an herbal tea product comprising oregano, peppermint, lungwort, sage, and licorice root, comprising:
bringing a volume of water to a gentle boil;
placing a tea bag containing the herbal tea product in the water;
setting the water and tea bag aside until the color of the tea water has a brown color;
pouring a portion of the colored tea water into a container; and
drinking the tea water from the container.

16. The method of claim 15, wherein the portion of colored tea water that is poured into the container amounts to approximately 16 ounces.

17. The method of claim 15, wherein the steps of claim 14 are repeated twice each day for two weeks and two containers of colored tea water are consumed each day for two weeks.

18. The method of claim 17, wherein the steps of claim 14 are repeated once each day thereafter and one container of colored tea water is consumed on each such day.

19. The method of claim 15, wherein the licorice root in the herbal tea product is crushed.

20. The method of claim 15, wherein the herbal tea product further includes at least one of OSHA root, eucalyptus, plantain leaf, chaparral, elecampane, lobelia, and thyme.

21. The method of claim 20, wherein any OSHA root or elecampane in the herbal tea product is crushed.
